(19) United States
(12) Patent Application Publication
(10) Pub. No.: US 2025/0002893 A1
(43) Pub. Date: Jan. 2, 2025

(54) Method for Improving Acetate Tolerance and Lipid Accumulation of Oleaginous Microorganism Using Acetyl-CoA Synthetase (ACS)

(71) Applicant: Jiangnan University, Wuxi (CN)

(72) Inventors: Haiqin Chen, Wuxi (CN); Lulu Chang, Wuxi (CN); Bo Yang, Wuxi (CN); Jianxin Zhao, Wuxi (CN); Wei Chen, Wuxi (CN)

(21) Appl. No.: 18/830,762

(22) Filed: Sep. 11, 2024

Related U.S. Application Data
(63) Continuation of application No. PCT/CN2023/091440, filed on Apr. 28, 2023.

(30) Foreign Application Priority Data
Dec. 19, 2022 (CN) 2022116348669

(51) Int. Cl. C12N 9/00; C12N 9/04; C12N 9/12; C12N 9/88; C12N 15/80; C12P 7/64

(52) U.S. Cl. CPC C12N 9/93; C12N 9/0006; C12N 9/12; C12N 9/88; C12N 15/80; C12P 7/64; C12Y 602/01001

(57) ABSTRACT

The present disclosure discloses a method for improving acetate tolerance and lipid accumulation of an oleaginous microorganism using an acetyl-CoA synthetase (ACS) and belongs to the technical fields of genetic engineering and microbial engineering. The present disclosure overexpresses endogenous constitutive MaACS in Mortierella alpina, improves the acetate tolerance and lipid yield of the recombinant strain, and the proportion of polyunsaturated fatty acids, and further improves the lipid accumulation using a culture medium taking ammonium acetate as a nitrogen source. The present expresses a citrate lyase ACL1 while overexpressing the MaACS in a combined manner, or expresses glucose-6-phosphate dehydrogenase G6PD2 to equilibrate the supply of reducing power NADPH so as to avoid the overflow of the acetyl-CoA, or knocks down a γ subunit SNF1 γ of a sucrose non-fermenting-related protein kinase which negatively regulates lipid accumulation to improve the activity of an acetyl-CoA carboxylase 1.

### REFERENCE TO SEQUENCE LISTING

The instant application contains a Sequence Listing in XML format as a file named “3050-YGHY-2023-63-SEQ.xml”, created on Sep. 10, 2024, of 24929 bytes in size, and which is hereby incorporated by reference in its entirety.

### TECHNICAL FIELD

The present disclosure relates to a method for improving acetate tolerance and lipid accumulation of an oleaginous microorganism using an acetyl-COA synthetase (ACS), specifically relates to an acetyl-CoA synthetase and use thereof in the production of an oil rich in polyunsaturated fatty acids, optimizes an acetyl-CoA metabolic pathway by a polygene operating system, improves the lipid accumulation capacity of Mortierella alpina, and belongs to the technical field of genetic engineering and microbial engineering.

### BACKGROUND

The intake level of polyunsaturated fatty acids (PUFAs) is an important indicator for evaluating the quality of diet. Human body mainly obtains the polyunsaturated fatty acids from deep sea fish and vegetable oil in diet. With the rise of the bio-manufacturing industry, oleaginous microorganisms become an important source of the dietary polyunsaturated fatty acids and mainly include Schizochytrium, Crypthecodinium cohnii and filamentous fungi (M. alpina and Mucor circinelloides). The C. cohnii is a main strain for commercially producing docosahexaenoic acid (DHA). The M. alpina is the only strain for commercially producing arachidonic acid (ARA) at present. Related oil products are mainly used as a nutrient supplements for infant formula.

In recent years, more and more lipid synthesis pathways and mechanisms of the oleaginous microorganisms are analyzed, relatively complete omics research methods and genetic operating systems are provided, and a more definite fermentation scheme design and genetic modification direction is provided for improving the yield of target fatty acids. In the production of PUFAs-rich oils using the oleaginous microorganisms, culture conditions and product yields are important considerations. Therefore, researchers mainly start with the selection of carbon and nitrogen sources, the improvement of production performance of strains and the yield of target products. The carbon and nitrogen sources with low cost are used as substrates for microorganism cell growth and lipid production. Relatively high carbon flow is converted into acetyl-CoA for fatty acid synthesis, which is the most effective way for improving the production performance of the strains and the economic benefit of the products.

Taking M. alpina as an example, when common nitrogen sources such as ammonium sulfate or ammonium tartrate are used for fermentation, the pH value of a culture solution is reduced to be 4 or below at the later stage of fermentation. However, it is proved in the previous stage that fatty acid desaturation and elongation processes are more suitably performed in an environment with the pH value of 6 or above. The over low pH value can inhibit the transformation of ultra-long-chain PUFAs and reduce the production performance of the strain and the economic benefit of the product. Currently, all fermentation technologies mainly automatically adjust or maintain the pH value of a fermentation solution by real-time detection on the fermentation solution, which limits the control and equipment of the fermentation process to a certain extent. In recent years, metabolomics research based on liquid chromatography-mass spectrometry shows that a small amount of acetic acid is produced and secreted extracellularly during the fermentation process of the oleaginous microorganisms such as M. alpina. However, acetate has a relatively strong biotoxicity. That is, 10 mmol/L of the acetate inhibits growth, consequently shortening a lipid accumulation period and affecting the product yield. Similar problems exist in the oleaginous alga C. cohnii.

Therefore, it is of important significance to optimize the nitrogen source in the M. alpina fermentation process, find a more convenient and efficient pH adjusting method for a fermentation solution, and improve tolerance of the microorganism to a metabolite for strengthening the lipid yield of the microorganism cells, reducing the fermentation cost and improving the proportion of the ultra-long-chain PUFAs with an economic benefit.

An acetyl-CoA synthetase (ACS), also known as an acetate-CoA ligase, participates in the following reactions: in the presence of ATP, an intermediate acetyl-AMP is first synthesized, and then the acetyl-AMP is linked with CoA in the form of a high-energy thioester bond to generate acetyl-CoA and simultaneously release one molecule of AMP and pyrophosphoric acid. Therefore, the ACS is also called an AMP-forming acetyl-CoA synthetase.

ATP+Acetate+CoA<=>AMP+PPi+Acetyl-CoA

The ACS is widely found in prokaryotes and eukaryotes and is an important node of lipid and acetate metabolism. It has been reported that overexpression of the ACS in bacteria results in substantial conversion of the acetate to the acetyl-CoA, thereby supporting the subsequent glycolytic pathway, fatty acid metabolism, amino acid metabolism, and gluconeogenesis, and accelerating cell growth and accumulation of acetyl-CoA derivatives. The most major research on the ACS has focused on its mechanism of affecting survival of tumor cells: in cancer cells with a poor supply of glucose and oxygen, the highly expressed ACS utilizes the byproduct of acetate to synthesize acetyl-CoA so as to activate a fatty acid synthesis pathway as an additional nutritional source, thereby maintaining the survival of the tumor cells. However, microbial ACS is less studied for its function in lipid metabolism. The reason is that compared with the glycolytic pathway using glucose as a carbon source, the ACS pathway is not the major source of intracellular acetyl-CoA in microorganisms.

The transcription of the ACS in the eukaryotes is regulated by an intracellular carbon source. A promoter of ACS1 is an inducible promoter, whose gene expression is inhibited in the presence of glucose but can be activated by the acetic acid (acetate) in the absence of the glucose. The transcription of ACS2 is regulated by its upstream constitutive promoter and is not affected by an external nutritional level. Similar phenomena also occur in bacteria. Besides, studies demonstrate that the ACS2 is also involved in transcriptional regulation of the ACS1. According to different species, the ACS further has different subcellular localizations. The ACS present in cytoplasm is involved in acetyl-CoA synthesis. The ACS present in mitochondria and peroxisomes is involved in fatty acid oxidation.

M. alpina is an oleaginous filamentous fungus having a strong lipid synthesis ability and is also currently the only commercial producer for the ARA. During the fermentation process, the pH value and the nitrogen source type have great influence on the lipid accumulation and the composition of the M. alpina. In addition, metabolomics data show that a small amount of acetic acid is generated at the final stage of fermentation of the M. alpina and is secreted extracellularly. The acetic acid reduces the utilization rate of the microorganism cells on glucose and also has certain influence on the growth of the microorganism cells. In addition, reports have shown that some cellulose/lignin hydrolysates are good carbon sources. However, since they contain toxic substances such as acetic acid and the like, the cellulose/lignin hydrolysates have higher requirements on strain tolerance and have limited application range. Therefore, it is urgently needed to find a method for improving the acetate tolerance and enhancing lipid accumulation and PUFAs yield of M. alpina, which is of important significance to further improve the production performance of the strain, widen the application range of carbon sources and stabilize culture conditions.

### SUMMARY

According to the analysis of genome data of M. alpina ATCC 32222, there are 2 AMP-forming ACS encoding genes Maacs1 and Maacs2 in the M. alpina, and the homology between the two genes is 85%. Bioinformatics analysis results show that the molecular weights of MaACS1 and MaACS2 are 72.2 kDa and 72.1 kDa, the isoelectric points are 6.51 and 6.62, and the main structural domains include an adenosine monophosphate (AMP) binding domain, a coenzyme A binding domain, an activation site and the like. Subcellular localization shows that the MaACS1 and MaACS2 are both cytoplasmic proteins without mitochondrial localization signal peptides. According to the transcriptome and proteome data of the M. alpina, both Maacs1 and Maacs2 can be transcribed and translated normally under the condition of taking glucose as the sole carbon source, but the transcription level of the Maacs2 and the expression level of the corresponding protein are significantly higher than those of the Maacs1. Therefore, the Maacs2 is presumed to be the ACS playing the main role in the M. alpina. However, there are few studies related to the functional identification of the ACS derived from oleaginous microorganisms such as M. alpina. There are no reports on the functional studies in the aspects of acetate utilization and lipid accumulation improvement.

The present disclosure provides an acetic acid-tolerant recombinant strain. M. alpina is used as a host for overexpressing an acetyl-CoA synthetase with an amino acid sequence shown in SEQ ID No: 1.

In one embodiment, the recombinant strain further knocks down or inhibits a γ subunit of a sucrose non-fermenting-related protein kinase, overexpresses a citrate lyase and/or overexpresses a glucose-6-phosphate dehydrogenase 2.

In one embodiment, an amino acid sequence of the citrate lyase is shown in SEQ ID No: 9, an amino acid sequence of the glucose-6-phosphate dehydrogenase 2 is shown in SEQ ID No: 10, and an amino acid sequence of the γ subunit of the sucrose non-fermenting-related protein kinase is shown in SEQ ID No: 11.

In one embodiment, a nucleotide sequence of a gene encoding the acetyl-CoA synthetase is shown in SEQ ID No: 2.

In one embodiment, the recombinant strain takes a pBIG2-ura5s-ITs plasmid as an expression vector.

In one embodiment, the pBIG2-ura5s-ITs vector is described in the patent application text with the publication No. CN103571762A.

In one embodiment, the pBIG2-ura5s-ITs plasmid carries a protein tag.

In one embodiment, the protein tag includes myc-tag with an amino acid sequence shown in SEQ ID No: 3 or flag with an amino acid sequence shown in SEQ ID No: 4.

In one embodiment, the recombinant strain takes M. alpina MA-Pcbh1-LbCpf1-ura5⁻ or CCFM 501 as a host.

In one embodiment, the M. alpina CCFM 501 is described in the patent application text with the authorization number of ZL 201310347934.8 and the M. alpina MA-Pcbh1-LbCpf1-ura5⁻ is described in the patent application text with the publication number of CN112592926A.

The present disclosure further provides a method for regulating an oleaginous

microorganism to synthesize a lipid, where an ammonium salt is used as a nitrogen source, 0-30 mmol/L of an acetate is added, and an acetyl-CoA synthetase with an amino acid sequence shown in SEQ ID No: 1 is overexpressed in the oleaginous microorganism.

In one embodiment, a concentration of the ammonium salt is 12-21 mmol/L.

In one embodiment, a concentration of the acetate is 12-30 mmol/L.

In one embodiment, the oleaginous microorganism includes M. alpina.

In one embodiment, the recombinant strain takes M. alpina MA-Pcbh1-LbCpf1-ura5- or CCFM 501 as a host.

In one embodiment, the M. alpina CCFM 501 is described in the patent application text with the authorization number of ZL 201310347934.8 and the M. alpina MA-Pcbh1-LbCpf1-ura5⁻ is described in the patent application text with the publication number of CN112592926A.

In one embodiment, the lipid includes triglycerides (rich in arachidonic acid), phospholipids, or free fatty acids.

The present disclosure further provides a whole-cell catalyst containing the recombinant strain.

In one embodiment, the present disclosure further provides a method for synthesizing a lipid, taking the recombinant strain or the whole-cell catalyst as a fermentation strain and synthesizing the lipid in a fermentation system containing an acetate and an ammonium salt.

In one embodiment, the fermentation system contains 20-80 g/L of glucose, 1-2 g/L of a yeast extract, 5-10 g/L of potassium dihydrogen phosphate, 1.5-2.5 g/L of disodium hydrogen phosphate, 1-2 g/L of magnesium sulfate heptahydrate, 0.08-0.12 g/L of calcium chloride dihydrate and trace elements, 12-21 mmol/L of the ammonium salt, and 0-30 mmol/L of the acetate.

In one embodiment, a concentration of the acetate is 12-30 mmol/L.

In one embodiment, the ammonium salt includes ammonium acetate and/or

ammonium tartrate.

In one embodiment, the acetate includes sodium acetate.

In one embodiment, the concentrations of the trace elements are as follows: 0.001 g/L of ferric chloride heptahydrate, 0.0001 g/L of zinc sulfate heptahydrate, 0.0001 g/L of copper sulfate pentahydrate, 0.0001 g/L of cobalt nitrate and 0.0001 g/L of manganese sulfate pentahydrate.

The present disclosure further provides use of the recombinant strain or the whole-cell catalyst or the method in the production of a lipid.

The present disclosure further provides a method for extracting total protein of an oleaginous microorganism, including grinding the recombinant strain under liquid nitrogen, using precooled acetone containing 10% trichloroacetic acid (TCA) to precipitate the microorganism cell to remove a metabolite, then using a urea extracting solution to extract a protein, and then adding iced acetone to precipitate a sample, thereby obtaining the total protein suitable for immunoblotting analysis.

In one embodiment, the method includes the following specific steps:
(1) taking a wet microorganism cell and grinding same into microorganism powder under liquid nitrogen;
(2) adding precooled 10% TCA/acetone (w/v), shaking and mixing the solution uniformly, then placing same overnight at −18˜−22° C. for precipitation, and carrying out centrifugation and removing a supernatant;
(3) adding precooled iced acetone, shaking and mixing the solution uniformly, then placing same at −18° C. to −22° C. again for precipitation for 1.5-2.5 h, carrying out centrifugation and removing a supernate, repeating the step for 2-3 times, and drying same until the acetone is completely volatilized;
(4) adding a urea extracting solution, fully shaking and dissolving the solution, placing same in a constant-temperature incubator at 26-30° C. for 0.5-1.5 h, uniformly mixing same once every other 15-30 min during the period, carrying out centrifugation, taking a supernatant, and then adding 1 mL urea extracting solution into the precipitate for fully extraction of remaining protein, repeating the step, and mixing the supernatant obtained each time
(5) adding iced acetone at 4.5-5.5 times of the volume of the supernatant into the supernatant, uniformly mixing the solution for precipitation for more than or equal to 1.5-2.5 h or precipitation overnight, carrying out centrifugation, washing the precipitate with the iced acetone for 2-3 times, drying and dissolving the precipitate using 8 M urea to obtain total protein solution.

In one embodiment, the urea extracting solution includes 150 mmol/L of Tris-HCl at the pH of 8.0, 6.0-9.0 mol/L of urea, 0.5-1.0% (w/v) of sodium dodecyl sulfate (SDS), 10-65 mmol/L of dithiothreitol (DTT) and a protease inhibitor (PMSF).

The present disclosure further provides use of the method in protein immunoassay detection of an oleaginous microorganism.

### Beneficial Effects

According to the research of the present disclosure, the addition of the acetate is beneficial to the lipid accumulation of M. alpina and the synthesis of the PUFAs, but 10 mmol/L of the acetate can inhibit growth of microorganism cells to a certain extent. In order to solve the problem, the present disclosure overexpresses the acetyl-CoA synthetase MaACS2 in M. alpina and obtains recombinant M. alpina with a stronger acetate tolerance by screening. After cultured for 48 h under the condition that 21 mmol/L of the ammonium acetate is used as a nitrogen source, the biomass of the recombinant M. alpina is 1.68-1.83 times (p<0.05) of that of a wild-type strain, the average total biomass at the end of fermentation is 13.4±0.5 g/L, which is 1.21 times of that of the wild-type strain, and the average total fatty acid yield is 5.9±0.3 g/L, which is 1.44 times of that of the wild-type strain. Since the ammonium acetate has a good pH buffering property, the natural pH value of a microorganism cell fermentation solution is stabilized at 6.3-6.5, the synthesis of the PUFAs is remarkably promoted, and the yield of arachidonic acid (ARA) in the MaACS2 recombinant strain is improved to 2.8 g/L, which is 5.3 times of that of the wild-type strain when ammonium tartrate is used as a nitrogen source. On the basis, a CRISPR/Cpf1 polygene operating system is used for further enhancing the supply of cytoplasmic acetyl-CoA (ACL1), enhancing the supply of reducing power NADPH (G6PD2) and indirectly increasing the activity of an acetyl-CoA carboxylase (ACC1) to promote lipid accumulation (SNF1-γ subunit interference). The lipid accumulation capacity of the M. alpina is optimized by optimizing acetyl-CoA synthesis and consumption pathways to balance supply of the acetyl-CoA, and guiding the acetyl-CoA to flow to a lipid synthesis pathway. The present solution further provides a protein extraction method suitable for the oleaginous microorganism. The function of the target gene can be more reasonably researched through the determination of a protein expression level by western blot and the influence of an insertion site on the protein expression level can be eliminated.

### BRIEF DESCRIPTION OF FIGURES

FIG. 1 shows a result of genome PCR verification of transformants of recombinant M. alpina Maacs2 (MA-Maacs2). M is marker; and N is a control group, namely prototrophic M. alpina transformed with an empty plasmid, and the rest is transformants.

FIG. 2 shows a result of preliminary screening of transformants of the recombinant M. alpina Maacs2 (MA-Maacs2), and the control group is wild-type M. alpina ATCC 32222. * represents that the data is significant compared to that of the control group and ns indicates no significance.

FIG. 3A shows growth at different time points of the recombinant M. alpina Maacs2 in a culture medium with ammonium acetate as a nitrogen source, and the control group is the wild-type M. alpina ATCC 32222. * represents the data is significant compared to that of the control group (the wild-type strain at the same time point) and ns indicates no significance.

FIG. 3B shows lipid production levels of the recombinant M. alpina Maacs2 in a culture medium with ammonium acetate as a nitrogen source, and the control group is the wild-type M. alpina ATCC 32222. * represents the data is significant compared to that of the control group (the wild-type strain at the same time point) and ns indicates no significance.

FIG. 4 shows expression levels of the target protein MaACS2 of the recombinant M. alpina Maacs2 at different time points in the culture medium with the ammonium acetate as the nitrogen source, and the control group is the wild-type M. alpina ATCC 32222(N).

FIG. 5A shows the total biomass of the recombinant strain during synergistic overexpression of Maacs2 and Mag6pd2.

FIG. 5B shows the total lipid production of the recombinant strain during synergistic overexpression of Maacs2 and Mag6pd2.

FIG. 5C shows the total biomass of the recombinant strain during synergistic overexpression of Maacs2 and Maacl1.

FIG. 5D shows the fatty acid content of the recombinant strain during synergistic overexpression of Maacs2 and Maacl1.

FIG. 5E shows the total biomass of the recombinant strain during overexpression of Maacs2 and RNA interference of Masnf4Ri.

FIG. 5F shows the fatty acid content of the recombinant strain during overexpression of Maacs2 and RNA interference of Masnf4Ri.

FIG. 6A-6B shows expressions of M. alpina MaACS1 and MaACS2 in Saccharomyces cerevisiae, where A shows verification of S. cerevisiae expression vector; and B shows protein expression levels of MaACS1 and MaACS2 in recombinant S. cerevisiae under different induction times and number 1-5 respectively represent induction at 24 h, 36 h, 48 h, 72 h and 96 h in a galactose system.

### DETAILED DESCRIPTION

M. alpina ATCC 32222 involved in the following examples is purchased from American Type Culture Collection (ATCC); Agrobacterium tumefaciens AGL-1 involved in the following examples is purchased from the Beijing Huayueyang Bio; Escherichia coli DH5α involved in the following examples is purchased from Invitrogen; a pBIG2-ura5s-ITs vector involved in the following examples is described in the patent application text with the publication number of CN103571762A; and pBIG2-ura5s-2myc-ITs involved in the following examples is obtained by linking 2 times of a myc protein fusion tag at the front end of Its on the basis of pBIG2-ura5s-ITs and the sequence of the myc protein fusion tag is shown in SEQ ID No: 3.

The following glucose-6-phosphate dehydrogenase G6PD2 is described in the patent application text with the publication number of CN105368727A, an amino acid sequence is shown in SEQ ID No: 10 and a nucleotide sequence is shown in SEQ ID No: 13; a subunit SNF1γ of a sucrose non-fermenting-related protein kinase γ and an interference vector are described in the patent application text with the publication number of CN110656097A, an amino acid sequence of the γ subunit SNF1γ of the sucrose non-fermenting-related protein kinase is shown in SEQ ID No: 11, and a nucleotide sequence is shown in SEQ ID No: 14; and an amino acid sequence of a citrate lyase ACL1 is shown in SEQ ID No: 9 and a nucleotide sequence is shown in SEQ ID No: 12.

A method for expressing polygenes in M. alpina, an engineered M. alpina strain MA-Pcbh1-LbCpf1-ura5⁻ with a polygene operating system, a uracil targeted knockout and recovery method and the related culture mediums are described in the patent application text with the publication number of CN112592926A.

A KOD plus high-fidelity DNA polymerase involved in the following examples is purchased from Toyobo, Japan; a taq DNA polymerase involved in the following examples is purchased from CWBIO; a reverse transcription kit (PrimeScript RT regent Kit with gDNA Eraser RR047A&R6110A) involved in the following examples is purchased from Takara, Japan; a plasmid extraction kit involved in the following examples is purchased from Tiangen Biotech (Beijing); a fungal genomic DNA extraction kit involved in the following examples is purchased from BioFlux; restriction enzymes, a T4 ligase, Trizol, a PCR product purification kit, a gel recovery kit, GeneRuler DNA Ladder Mix and PageRuler Prestained Protein Ladder involved in the following examples are all purchased from Thermo Scientific; n-pentadecanoic acid (C15:0), ammonium acetate, sodium acetate and microcrystalline cellulose involved in the following examples are all purchased from Sigma; DEPC water, kanamycin (Kana), rifampicin (Rif), spectinomycin (Spe), cefotaxime sodium (Cef), a yeast nitrogen base without amino acids (YNB), and various amino acids involved in the following examples are purchased from Sangon Biotech (Shanghai); a yeast extract and tryptone involved in the following examples are purchased from Oxoid; low adsorption-type enzyme-free pipette tips, enzyme-free centrifuge tubes, enzyme-free PCR tubes, and 2-mL brown chromatography vials and vial lids involved in the following examples are purchased from Suzhou Keqing biological company; a transformation inducer acetosuringone (AS, CAS #[2478-38-8]), 2-(N-morpholino) ethanesulfonic acid (MES buffer, CAS #[145224-94-8]), uracil, 5-fluorouracil, a yeast nitrogen base without amino acids (CAS #[A610507-0500] Lot: C418BA0040) and various amino acids involved in the following examples are purchased from Sangon Biotech (Shanghai); and other reagents are purchased from Sinopharm Group.

The vector construction and the preparation of competent cells of bacteria involved in the following examples all refer to Molecular Cloning.

The primer and sequencing work involved in the following examples are performed by BGI (Shanghai).

Culture mediums involved in the following examples are as follows:

Broth medium: 20 g/L (used during activation)/30 g/L of (used during lipid production) glucose, 5 g/L of a yeast extract, 1 g/L of potassium dihydrogen phosphate, 0.25 g/L of magnesium sulfate heptahydrate and 10 g/L of potassium nitrate.

Kendrick medium: 20 g/L (used during activation)/30 g/L of (used during lipid production) glucose, 3.3 g/L of ammonium tartrate (used during activation)/1.67 g/L of ammonium acetate (used during lipid production), 1.5 g/L of a yeast extract, 7 g/L of dipotassium hydrogen phosphate, 2.0 g/L of potassium dihydrogen phosphate, 1.5 g/L of magnesium sulfate heptahydrate and 0.1 g/L of calcium chloride dihydrate and trace elements,

where the concentrations of the trace elements are as follows: 0.001 g/L of ferric chloride heptahydrate, 0.0001 g/L of zinc sulfate heptahydrate, 0.0001 g/L of copper sulfate pentahydrate, 0.0001 g/L of cobalt nitrate and 0.0001 g/L of manganese sulfate pentahydrate.

Ammonium nitrogen in the Kendrick medium (used during lipid production) can be replaced by 2.0 g/L of ammonium tartrate instead of 1.67 g/L of ammonium acetate, and 12-30 mmol/L of acetate (sodium acetate) is additionally added.

GY medium: 20 g/L of glucose, 10 g/L of a yeast extract, 2 g/L of potassium nitrate, 1 g/L of sodium dihydrogen phosphate, 3 g/L of magnesium sulfate heptahydrate and 20 g/L of agar powder.

GY auxotrophic strain selection medium (GYU-F) for screening uracil knockout auxotrophic strain: on the basis of the GY medium, 0.5 g/L of 5-fluorouracil (5-FOA) and 0.1 g/L of uracil are added, where 5-FOA is dissolved by dimethyl sulfoxide, sterilized by filtration using a 0.22-μm organic filter membrane, added after the sterilization and cooling, and stored and used in a dark place.

MM induction and knockout medium (induction of Pcbh1 promoter and nuclease and knockout of uracil screening marker): 3 g/L of microcrystalline cellulose, 4 g/L of potassium dihydrogen phosphate, 2.8 g/L of ammonium sulfate, 0.6 g/L of magnesium sulfate heptahydrate, 0.5 g/L of calcium chloride, 0.6 g/L of urea, 3 g/L of tryptone, 1 ml/L of Tween 80, 5 g/L of calcium carbonate, 0.01 g/L of ferrous sulfate heptahydrate, 0.0032 g/L of manganese sulfate monohydrate, 0.0028 g/L of zinc sulfate heptahydrate and 0.004 g/L of cobalt chloride. MM minimum medium: 1.74 g/L of dipotassium hydrogen phosphate, 1.37 g/L of potassium dihydrogen phosphate, 0.146 g/L of sodium chloride, 0.49 g/L of magnesium sulfate heptahydrate, 0.078 g/L of calcium chloride, 0.53 g/L of ammonium sulfate, 1.8 g/L of glucose, 10 mL/L of ferric sulfate heptahydrate (100×), and 5 mL/L of glycerol. After sterilization, a microorganism-filtered MES buffer is added to a final concentration of 7.8 g/L.

IM induction medium (for A. tumefaciens-mediated transformation): slightly adjusted on the basis of the MM culture medium, additionally added with 0.1 g/L of uracil, glucose changed to 0.9 g/L, and the rest kept unchanged. Before use, 100 μg/mL of acetosyringone (AS) and 7.8 g/L of MES are added, 20 g/L of agar strips are added when a solid culture medium is prepared, and the IM culture medium containing the AS needs to be stored in a dark place.

SC screening medium: 20 g/L of glucose, 5 g/L of a yeast nitrogen base without amino acids, 1.7 g/L of ammonium sulfate, 10 mL/L of an amino acid mother solution (100×), and 20 g/L of agar.

SC-CS medium (for selection of transformants): 20 g/L of glucose, 5 g/L of a yeast nitrogen base without amino acids, 1.7 g/L of ammonium sulfate, 10 mL/L of an amino acid mother solution (100×), and 20 g/L of agar. Before poured onto a plate, 100 μg/mL of cefotaxime and 100 μg/mL of spectinomycin are added,

where the amino acid mother solution are as follows: 60 mg/L of isoleucine, 60 mg/L of leucine, 60 mg/L of phenylalanine, 50 mg/L of threonine, 40 mg/L of lysine, 30 mg/L of tyrosine, 20 mg/L of adenine, 20 mg/L of arginine, 20 mg/L of histidine and 10 mg/L of methionine.

SOC recovery medium: 20 g/L of tryptone, 5 g/L of a yeast extract, 0.5 g/L of sodium chloride, 0.186 g/L of potassium chloride, 0.95 g/L of magnesium chloride and 3.6 g/L of glucose.

LB liquid medium: 10 g/L of tryptone, 5 g/L of a yeast extract and 10 g/L of sodium chloride. Before use, 100 μg/mL of kanamycin is added.

LB solid medium: 10 g/L of tryptone, 5 g/L of a yeast extract, 10 g/L of sodium chloride and 20 g/L of agar. Before use, 100 μg/mL of kanamycin is added.

YEP liquid medium: 10 g/L of a yeast extract, 10 g/L of trypsin and 5 g/L of sodium chloride. Before use, 100 μg/mL of kanamycin and 100 μg/mL of rifampicin are added, and the mixture is stored in a dark place.

YEP solid medium: 10 g/L of a yeast extract, 10 g/L of trypsin, 5 g/L of sodium chloride and 20 g/L of agar. Before use, 100 μg/mL of kanamycin and 100 μg/mL of rifampicin are added, and the mixture is stored in a dark place.

Protein extracting solution: 8 mol/L of urea, 1% (w/w) of sodium dodecyl sulfate, 65 mmol/L of dithiothreitol, 150 mmol/L of Tris-HCl at the pH of 8.0, and 0.1% (v/v) of a protease inhibitor PMSF.

### Example 1 Screening and Cloning of Gene Encoding Acetyl-CoA Synthetase

The specific steps were as follows:

according to the EC number (6.2.1.1) of an acetyl-CoA synthetase, screening and BLAST comparison were performed in a gene bank of M. alpina ATCC 32222 subjected to whole genome sequencing and splicing. Alternative genes capable of being normally transcribed and expressed according to transcriptome and proteome information were screened, secondary comparison screening was performed in an NCBI library, a finally obtained target gene was named as Maacs2 (a nucleotide sequence was shown in SEQ ID No: 2), and a protein coded thereby was named as MaACS2 (an amino acid sequence was shown in SEQ ID No: 1).

There were 2 AMP-forming ACS encoding genes Maacs1 and Maacs2 in the M. alpina, and the homology between the two genes was 85%. Bioinformatics analysis results showed that the Maacs1 and Maacs2 were respectively encoded by 1,980 and 1,968 bases, the molecular weights of the corresponding proteins were 72.2 kDa and 72.1 kDa respectively, the isoelectric points were 6.51 and 6.62, and the main structural domains included an adenosine monophosphate (AMP) binding domain, a coenzyme A binding domain, an activation site and the like. Subcellular localization showed that the two proteins were both located in the cytoplasm without mitochondrial localization signal peptides. According to the transcriptome and proteome data of the M. alpina, both Maacs1 and Maacs2 can be transcribed and translated normally under the condition of taking glucose as the sole carbon source.

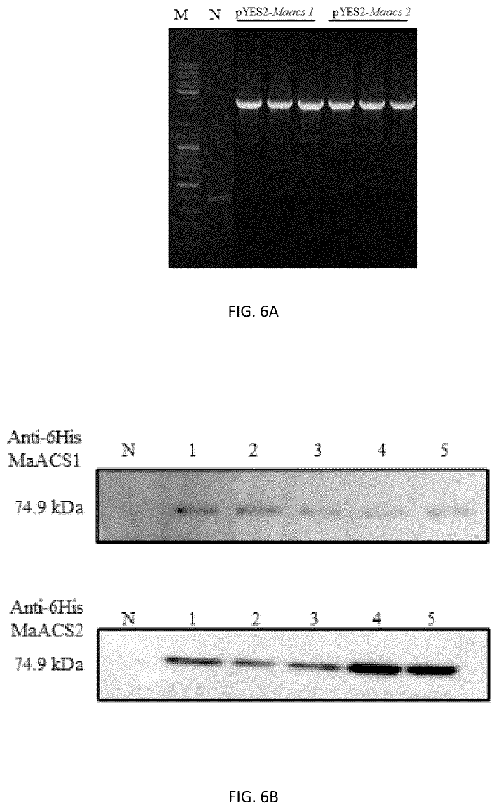

Heterologous expression of the Maacs1 and the Maacs2 was performed using a pYES2 series vector (pYES2-NT/C) of a commercial yeast expression system, a plasmid carrying the Maacs1 and the Maacs2 was transferred into S. cerevisiae, and induction culture was performed for 24 h-96 h. After the induction, the S. cerevisiae was centrifuged at 3,500 g for 5 min to collect the microorganism cells, which were stored in a refrigerator at −80° C. for a subsequent experiment (FIG. 6A).

The yeast after the induction expression was centrifuged to collect the microorganism cells and subpackaged as required, acid-washed glass beads and 0.5 mL of a yeast lysate (containing a protease inhibitor) were added, the mixture was intermittently oscillated at 65 Hz for 20 s for 5 times using a high-throughput tissue crusher and centrifuged at 10,000 g for 10 min, a supernatant was taken, and the protein concentration was measured using a BCA method or a Coomassie brilliant blue method (Bradford).

By expressing the two potential acetyl-CoA synthetase encoding genes in the S. cerevisiae, MaACS2 also showed a higher expression level, while the expression level of MaACS1 was extremely low under each induction condition (FIG. 6B). Besides, the transcription level of the Maacs2 was significantly higher than that of the Maacs1. The Maacs2 was presumed to be the ACS playing the main role in the M. alpina, such that the Maacs2 was selected as a research object for a subsequent analysis.

The total RNA of the M. alpina ATCC 32222 was extracted by a Trizol method, a cDNA was obtained by reverse transcription according to the instructions of a Takara reverse transcription kit. The Maacs2 was amplified by a PCR reaction in a cDNA library of the M. alpina ATCC 32222 with Maacs2 F/Maacs2 R as PCR amplification primers (Table 1). A PCR instrument was BIO-RAD T100 Thermal Cycler, a KOD plus high-fidelity DNA polymerase was used, a reaction system was 50 μL, and the system content was performed according to the instruction content of the DNA polymerase. The reaction process was as follows: pre-denaturing at 95° C. for 5 min, then denaturing at 95° C. for 30 s, annealing at 55° C. for 30 s, extending at 68° C. for 2.5 min, repeating the three steps for 32 times, then fully extending at 68° C. for 7 min, finally cooling to 12° C. and keeping for 10 min, and terminating.

After the reaction was finished, an amplification product was obtained, the amplification product was purified, and the band size of the amplification product was verified through a 1% agarose gel electrophoresis so as to obtain a DNA sequence of the Maacs2.

TABLE 1
Primer sequence and use thereof

| Name of | | |
|---|---|---|
| primers | Primer sequence (5′-3′) | Use |
| Maacs2 F | SEQ ID No: 5: | Used for |
| | CCCAAGCTTATGTCTGCTGAAGATACCGGCTAC | amplification of |
| Maacs2 R | SEQ ID No: 6: | Maacs2 gene |
| | TCCCCCGGGTTAAGAGCTGGCGACTTT | |

### Example 2 Expression of Maacs2 in M. alpina

The specific steps were as follows:

### (1) Construction of M. alpina Expression Vector

The DNA sequence of the Maacs2 obtained in example 1 and a vector pBIG2-ura5s-2myc-ITs were respectively cleaved with restriction enzymes Hind III and Sma I and ligated with a T4 ligase to obtain a ligation product. A specific enzyme digestion system (20 μL) was shown in Table 2.

TABLE 2
Enzyme digestion system

| Reagent | Amount |
|---|---|
| 10 × cutmart buffer | 2 μL |
| Restriction enzyme | 1 μL |
| PCR product or vector | 200 ng-1 μg |
| ddH₂O | supplemented to |
| | 20 μL |

After the ligation product was ligated overnight at 4-16° C., the ligation product was transformed into E. coli DH5α competent cells by the following transformation method: 100 μL of the competent cells were taken under the aseptic condition and 5-8 μL of the ligation product was added and uniformly mixed by pipetting; the uniformly mixed competent cells were transferred into a precooled electroporation cuvette to avoid generating bubbles; the electroporation cuvette was put into a Bio-Rad electroporation instrument, a proper preset program gear was adjusted, and electroporation was performed under the voltage condition of 1.8 kv; 1 mL of an SOC recovery medium was added into the electroporated competent cells, uniformly mixed, and transferred into a 1.5-mL centrifuge tube, and the cells were incubated at 37° C. and 150 rpm for 1 h; 200 μL of an LB solid medium plate coated with 100 μg/mL of kanamycin was taken and invertedly cultured overnight at 37° C.; and a positive transformant was picked and a plasmid was extracted. A sequencing verification result showed that the ligation was successful and a recombinant plasmid pBIG2-ura5s-2myc-Maacs2 was obtained.

### (2) Transformation Screening of M. alpina

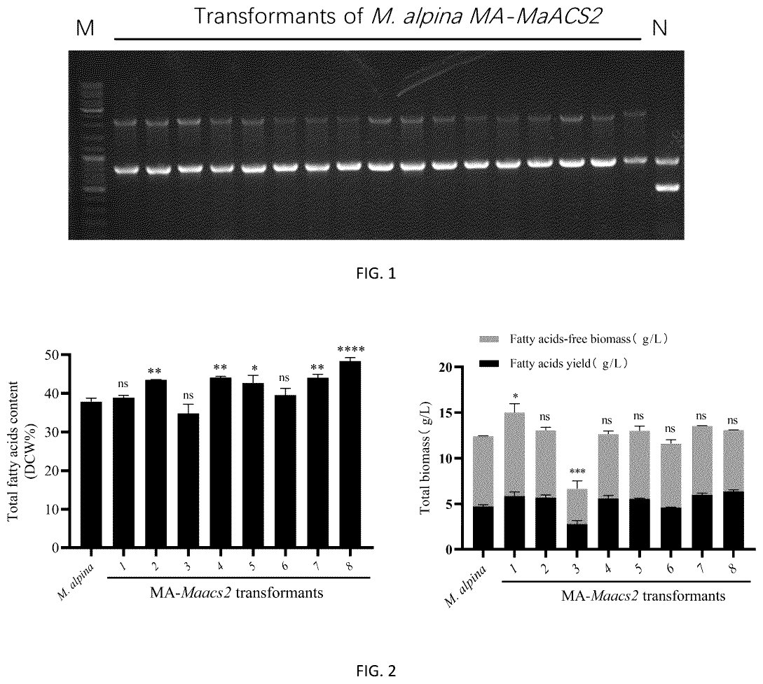

The recombinant plasmid pBIG2-ura5s-2myc-Maacs2 obtained in step (1) was transferred into A. tumefaciens AGL-1 by an electric shock transformation method to obtain the A. tumefaciens carrying the recombinant plasmid pBIG2-ura5s-2myc-Maacs2. The A. tumefaciens carrying the recombinant plasmid pBIG2-ura5s-2myc-Maacs2 was activated in a YEP liquid medium, cultured in an MM basal medium and induced in an IM induction medium respectively, the A. tumefaciens after the induction culture by the IM induction medium was taken and OD₆₆₀ was measured, and the bacteria were gradiently diluted to the OD₆₆₀=0.2-1.2 using the IM induction medium to obtain an A. tumefaciens liquid carrying the recombinant plasmid pBIG2-ura5s-2myc-Maacs2. Spores of M. alpina MA-Pcbh1-LbCpf1-ura5⁻ were scraped using normal saline, and placed in an incubator at 4-28° C. for 6-24 h to obtain germinated spore liquid, and the concentration of the spores was adjusted to 10⁷/mL. 100-200 μL of the A. tumefaciens liquid carrying the recombinant plasmid pBIG2-ura5s-2myc-Maacs2 and 100-200 μL of the M. alpina MA-Pcbh1-LbCpf1-ura5⁻ spore liquid were uniformly mixed upside down in a sterile EP tube, and the mixture was coated on an IM solid medium pasted with a glass paper and co-cultured at 16-28° C. for 12-48 h in a dark place. After the co-culture was finished, the glass paper was transferred to an SC-CS medium containing spectinomycin (Spe) and cefotaxime (Cef) and cultured at 16-28° C. until colonies grew. After the colonies grew, newly growing hyphae at the edges of the colonies were picked and continuously cultured on a new SC-CS medium at 28° C. for 12-48 h for subculture. After the subculture, the colonies capable of stably growing were picked to a Broth medium (used during activation, containing 20 g/L of glucose) and cultured at 28° C. for 2 d to obtain a microorganism liquid. The microorganism liquid was taken, fungal genome DNA was extracted to perform PCR verification, target bands were obtained by amplification, and a transformant simultaneously carrying a screening marker ura5s and a target gene Maacs2 was a correct positive transformant (FIG. 1), that is a recombinant M. alpina MA-Maacs2 carrying the Maacs2 gene was obtained, where the PCR was performed using a Taq enzyme system and the used primers are universal primers of a plasmid vector pBIG2-ura5s-ITs whose specific sequences were as follows:

upstream primer Hispro F1:
(SEQ ID No: 7)
CACACACAAACCTCTCTCCCACT;
and
downstream primer TrpCR 1:
(SEQ ID NO: 8)
CAAATGAACGTATCTTATCGAGATCC.

As can be seen from FIG. 1, PCR verification was performed using the universal primers, a uracil complementing marker ura5s of a T-DNA region of A. tumefaciens carrying the recombinant plasmid pBIG2-ura5s-2myc-Maacs2 and the target gene Maacs2 can be successfully amplified, and the band sizes conformed to the theoretical value (the obtained fragment was 156 bp longer than the actual fragment due to the design of the primers on the vector), which indicated that the target gene was successfully transferred into M. alpina and the transformant was named as MA-Maacs2.

Monospores of the recombinant M. alpina MA-Maacs2 transformant were randomly selected, inoculated into a Broth medium (used during activation, containing 20 g/L of glucose), and cultured at 28° C. for 2 d for activation. The microorganisms were continuously activated for three generations and centrifuged, and the activated microorganism cells were collected. The microorganism cells were crushed into uniform microorganism floccules. The crushed microorganism cells were inoculated into a Kendrick medium (during lipid production, containing 30 g/L of glucose) containing 21 mmol/L of ammonium acetate at the inoculation amount of 1% (v/v), and cultured under shaking at 28° C. and 200 rpm. The samples were respectively taken at 48 h, 96 h and 7 d. The culture medium was removed by suction filtration using a Buchner funnel, the microorganism cells were ground under liquid nitrogen, the powder was weighed to a 1.5-mL centrifugal tube, a crude enzyme extracting solution was added, the mixture was subjected to vortex oscillation, fully crushed and centrifuged, and the precipitate was discarded so as to obtain a supernatant. The supernatant containing a transformant of the acetyl-CoA synthetase MaACS2 was sequenced and the transformant with correct sequencing was selected for the next screening.

### Example 3 Growth and Lipid Production Analysis of Recombinant M. alpina

The specific steps were as follows:

### (1) Primary Screening

The recombinant M. alpina MA-Maacs2 transformant correctly sequenced in example 2 was subjected to primary screening, monospores of the transformant were respectively picked and inoculated into a Broth medium (used during activation, containing 20 g/L of glucose), and cultured at 28° C. for 2 d for activation. The microorganisms were continuously activated for three generations and centrifuged, and the activated microorganism cells were collected. The microorganism cells were crushed into uniform microorganism floccules. The crushed microorganism cells were inoculated into a Broth medium (during lipid production, containing 30 g/L of glucose) for primary screening at the inoculation amount of 1% (v/v), and cultured under shaking at 28° C. and 200 rpm for 7 d. 100 mL of the microorganism cells were collected using a Buchner funnel. The microorganism cells were freeze-dried in vacuum to the constant weight, the microorganism cells were weighed, and the biomass was calculated.

The microorganism cells were ground into powder, 30.00 mg of the microorganism powder was accurately weighed, 100 μL of 2 mg/mL of C15:0 was accurately added as an internal standard, and 2 mL of 4 mol/L hydrochloric acid was added to be fully and uniformly mixed. The mixture was subjected to water bath at 80° C. for 1 h and placed at −80° C. for 15 min. The step was repeated for 3 times, the mixture was cooled to room temperature, 1 mL of methanol and 1 mL of chloroform were added, uniformly mixed, and shaken for 2 min. The mixture was centrifuged at 3,000 g for 10 min. The chloroform layer was collected in a new lipid-extracting bottle. The step was repeated twice. The chloroform layers were combined and dried by blowing nitrogen, 1 mL of 10% of an anhydrous methanol solution containing 2% of sulfuric acid was added, and the mixture was subjected to a methyl esterification treatment in water bath at 70° C. for 4 h. Then 1 mL of saturated sodium chloride and 1 mL of n-hexane were added into the methyl esterification-treated system, uniformly mixed, and centrifuged at 2,500 g for 10 min, and the step was repeated twice. The n-hexane layer was collected in a new bottle, 1 mL of the n-hexane was continuously added into the residual liquid, and the mixture was shaken and uniformly mixed for 1 min, and centrifuged at 2,500 g for 10 min. The n-hexane layers were combined and dried by blowing nitrogen, and 1 mL of the n-hexane was added for redissolution to obtain a fatty acid methyl ester. The composition and the content of fatty acid in the microorganism cells were detected using GC-MS.

The fatty acid methyl ester was analyzed using GCMS-QP2010 Ultra (Shimadzu Co., Japan), chromatographic column: Rtx-Wax (30 m×0.25 mm, 0.25 μm) (Agilent Technologies, United States); ion source ionization mode: electron impact ionization (EI); ion source temperature and detector temperature: 220° C. and 250° C. respectively; sample injection temperature: 240° C., sample injection of 1 μL in a separation mode, split ratio: 10:1, and carrier gas: nitrogen; and temperature programming: maintaining the initial temperature at 150° C. for 2 min, increasing the temperature to 190° C. at 10° C./min, maintaining the temperature for 4 min, then increasing the temperature to 220° C. at 5° C./min, and maintaining the temperature for 6 min. By comparing and analyzing the peak area with that of the internal standard C15:0, the fatty acid component was quantified relatively and the total fatty acid content was expressed as the mass of the total fatty acids per microorganism cell.

As shown in FIG. 2, after the recombinant strain was cultured in the Broth fermentation medium containing potassium nitrate as a nitrogen source for 7 d, overexpression of the Maacs2 resulted in a varying degree of improvement in lipid accumulation. The total biomass was 12.42±0.108 g/L and the fatty acid content was 37.85±0.66 DCW % after the control group wild-type ATCC 32222 was fermented for 7 d. The fatty acid content of a part of the transformants was significantly higher than that of the control group wild-type ATCC 32222 (p<0.05). The fatty acid content of the transformant No. 2 and the transformant No. 8 respectively reached 43.53±0.05% and 48.37±0.62% of the dry cell weight. The fatty acid yield of the 2 transformants respectively reached 5.68±0.20 g/L and 6.34±0.15 g/L, and the two transformants were respectively named as MA-Maacs2-1 and MA-Maacs2-2 in a later experiment.

### (2) Growth and Lipid Production Analysis of Recombinant M. alpina

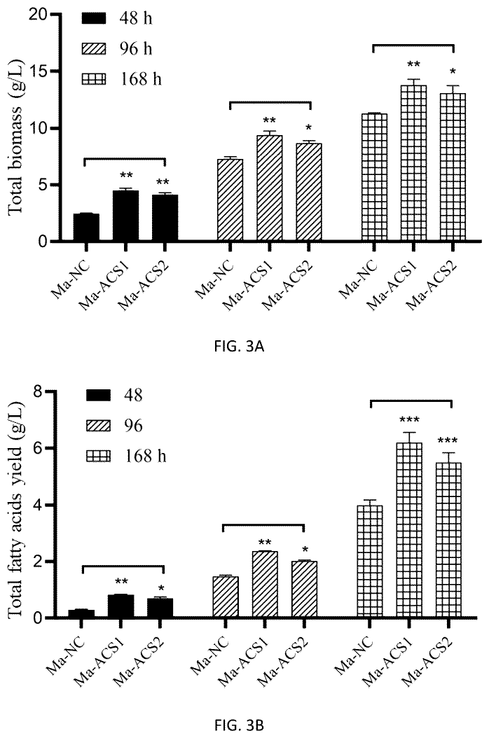

In order to further analyze the acetate tolerance and the growth and lipid accumulation at different time points of the recombinant strain, two recombinant strains (No. 2 and No. 8 transformants, marked as Ma-Maacs2-1 and Ma-Maacs2-2 in FIG. 3A-3B) screened in step (1) were selected. The activation process was the same as step (1), after the continuous activation for three generations, the crushed microorganism cells were inoculated into a Kendrick medium (used during lipid production, containing 30 g/L of glucose) at the inoculation amount of 1% (v/v), the microorganism liquid at the time points were respectively collected after the inoculation for 48 h, 96 h and 7 d, and the biomass and the fatty acid were analyzed. The result was shown in FIG. 3A and FIG. 3B.

According to the analysis of the sample growth and lipid production at different time points of the recombinant M. alpina in the Kendrick medium with ammonium acetate as a nitrogen source, the total biomass and the total fatty acid yield of the Maacs2 overexpression strain were significantly higher than those of the control group (p<0.05) at each stage of fermentation. At the initial stage of fermentation (48 h), the two recombinant strains grew well in 21 mmol/L of ammonium acetate, and the dry cell weight reached 4.14-4.50 g/L at 48 h, which was 1.68-1.83 times (p<0.05) of the control group wild-type strain ATCC 32222. The biomass advantage existed continuously in the fermentation process, and after 7 d of the fermentation culture, the biomass of the two recombinant strains respectively reached 13.80±0.03 g/L and 13.08±0.46 g/L, which was 1.22 times and 1.16 times respectively of that of the control group. After the two recombinant strains were used for fermentation for 7 d, the total fatty acid yield was 6.21±0.25 g/L and 5.50±0.24 g/L, which was 1.37 times and 1.55 times respectively of that of the control group. The results indicated that the overexpression of the Maacs2 in the M. alpina significantly improved the acetate tolerance of the microorganism cells, thereby promoting the strain growth and lipid accumulation. Since the ammonium acetate had a good pH buffering property, the natural pH value of a microorganism cell fermentation solution was stabilized at 6.3-6.5, the synthesis of the PUFAs was facilitated, the content of arachidonic acid (ARA), the representative PUFA, in the recombinant M. alpina MA-Maacs2 accounted for 46.8±1.25% of the total fatty acids, and the yield reached 2.8 g/L, which was 5.3 times of that of the wild-type strain ATCC 32222 under the culture condition of the Kendrick medium (not containing acetate) using ammonium tartrate as a nitrogen source.

### Example 4 Analysis of Protein Expression Level of Recombinant M. alpina

The specific steps were as follows:

The two recombinant strains in example 3 were selected, the microorganism liquid subjected to fermentation culture under the condition of the Kendrick medium using ammonium acetate as a nitrogen source at the time points of 48 h, 96 h and 7 d in step (2) of example 3 was obtained, the wet microorganism cells were taken and ground under liquid nitrogen, about 200 mg of the microorganism powder ground under liquid nitrogen was taken, zirconium oxide crushing beads were added, 1 mL of precooled 10% TCA/acetone (w/v) was added, the mixture was shaken, uniformly mixed, placed overnight at 20° C. for precipitation, and centrifuged at 12,000 g and 4° C. for 15 min, the supernatant was discarded, 1 mL of precooled iced acetone was added, and the mixture was shaken, uniformly mixed, and placed at −20° C. for precipitation for 2 h for washing away TCA. The mixture was centrifuged at 12,000 g and 4° C. for 15 min, the step was repeated 2 times until the TCA was completely washed away, and the precipitate was dried in a vacuum rotary dryer until acetone was completely volatilized.

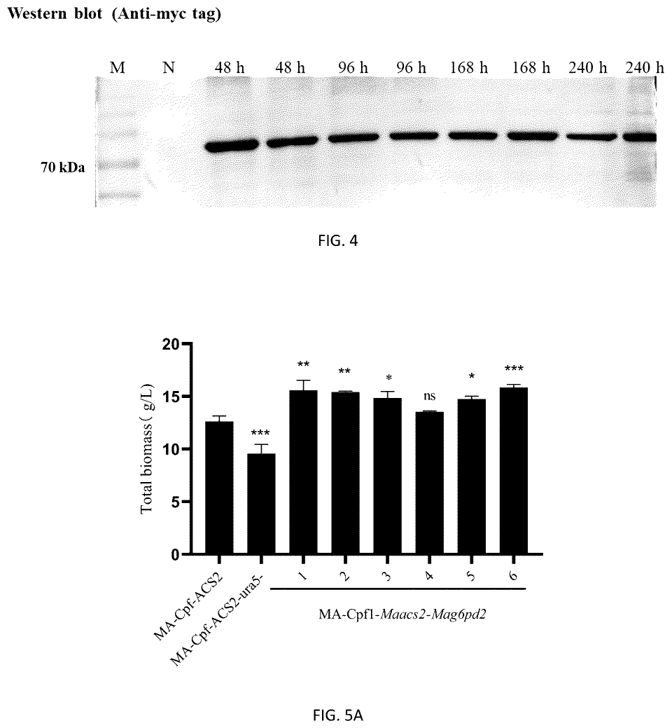

1 mL of a urea extracting solution was into the dried sample, fully shaken and dissolved, placed in a constant-temperature incubator at 28° C. for 1 h, and uniformly mixed once every other 20 min. The extracting solution was centrifuged at 12,000 g for 15 min at normal temperature, the precipitate was discarded, the supernatant was taken, 1 mL of the urea extracting solution was continuously added into the precipitate for repeated extraction once, after the supernatants were combined, iced acetone at 5 times of the volume of the combined supernatant was added and uniformly mixed, the mixture was precipitated for more than or equal to 2 h or precipitated overnight, centrifuged at 12,000 g for 15 min, washed with the iced acetone for 2 times to remove DTT and SDS, the residue was dried using a vacuum rotary instrument and dissolved using 8 M urea, the protein concentration was determined using a BCA method, and SDS-PAGE and western blot analysis were performed. SDS-PAGE, western blot and development method were performed according to the conventional procedures of “Protein Operation Manual”. The expression levels of the target protein in the recombinant M. alpina MA-Maacs2 at different time points were shown in FIG. 4.

According to the expression levels of a MaACS2 protein in the recombinant M. alpina MA-Maacs2 at different time points, it can be seen that the expression level of the target protein was slightly higher at 48 h, which corresponded to the fact that the recombinant strain grew faster and the fatty acid accumulation amount was higher at 48 h. The protein expression levels of the MaACS2 at other time points were basically consistent, which indicated that the MaACS2 was continuously expressed during the whole fermentation process, and no obvious protein degradation occurred after nitrogen source depletion at the later stage of fermentation. The present solution also provided a reliable technical method for protein expression analysis and related sample preparation in oleaginous filamentous fungi such as M. alpina, and provided a more objective reference basis for verifying the availability of the target gene in the genetically modified strain on the protein level.

### Example 5 Optimization of Acetyl-CoA Metabolic Pathway in M. alpina on the Basis of Polygene Combination

The specific steps were as follows:

At the previous stage, the M. alpina MA-Maacs2 was used as a starting strain to perform induction knockout of a screening marker ura5 gene using a gene editing technology (targeted induction knockout and screening steps of uracil screening marker referred to a patent application document with the publication number of CN112592926A).

Monospores of the starting strain MA-Maacs2 was inoculated into a Broth medium (used during activation, containing 20 g/L of glucose), and cultured at 28° C. for 2 d for activation. The microorganisms were continuously activated for three generations and centrifuged, and the activated microorganism cells were collected. The microorganism cells were crushed into uniform floccules. The crushed microorganism cells were inoculated into an MM induction and knockout medium at the inoculation amount of 1% (v/v), and cultured under shaking at 28° C. and 200 rpm for 7 d, 1 mL of the microorganism liquid was pipetted in a super clean bench and centrifuged to remove the supernatant, and the precipitate was coated on a GYU-F plate and stood and cultured at 28° C. in a dark place for 2 d, and then a uracil-deficient strain gradually grew out.

The new hyphae at the edges of the uracil-deficient strain were picked to a GYU-F screening plate for continuous passage for 3 times, and were synchronously inoculated in an SC screening medium without uracil for reverse verification (the deficient strain cannot grow on an SC screening plate without uracil), and the genome DNA extracted from the correct strain was verified using the universal primers (SEQ ID No: 7 and SEQ ID No: 8), and a uracil band cannot be amplified. Meanwhile, the uracil-deficient strain was subjected to fermentation culture. After the biomass and the fatty acid synthesis ability thereof were verified to be not different, an auxotrophic strain MA-2myc-Maacs2-Δura5⁻ deficient in the uracil screening marker was obtained again and used as a new starting strain for the introduction of the next gene.

In order to further improve the lipid accumulation capacity of the recombinant strain, referring to the step of expressing the Maacs2 in example 2, and combining the functional characteristic of the MaACS2, ACL1 was overexpressed in the MA-2myc-Maacs2-Δura5⁻ recombinant strain with the uracil screening marker knocked out, so as to further enhance the acetyl-CoA supply. G6PD2 was overexpressed to equilibrate reducing power (NADPH) to avoid acetyl-CoA overflow resulting in protein acetylation modification. A γ subunit SNF1γ of a sucrose non-fermenting-related protein kinase was interfered so as to improve the activity of an acetyl-CoA carboxylase (ACC1). The recombinant strains obtained in the present example were respectively named as MA-Cpf1-Maacs2-Maacl1, MA-Cpf1-Maacs2-Mag6pd2 and MA-Cpf1-Maacs2-Masnf4Ri.

Monospores of the recombinant M. alpina MA-Cpf1-Maacs2-Maacl1, MA-Cpf1-Maacs2-Mag6pd2 and MA-Cpf1-Maacs2-Masnf4Ri transformants correctly sequenced were selected, respectively inoculated into the Broth medium (used during activation, containing 20 g/L of glucose), and cultured at 28° C. for 2 d for activation. The microorganisms were continuously activated for three generations and centrifuged, and the activated microorganism cells were collected. The microorganism cells were crushed into uniform microorganism floccules. The crushed microorganism cells were inoculated into a Kendrick medium (during lipid production, containing 30 g/L of glucose) containing 21 mmol/L of ammonium acetate at the inoculation amount of 1% (v/v), the microorganism liquid was collected after the inoculation for 7 d, and the biomass and the fatty acid were analyzed. The result was shown in

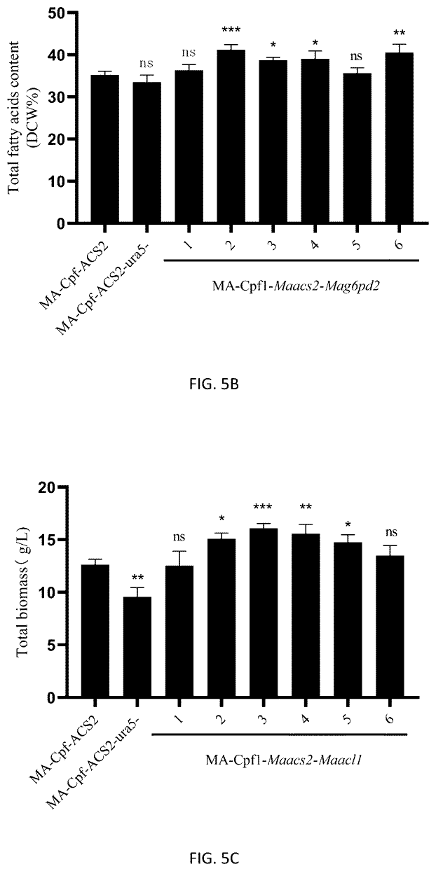

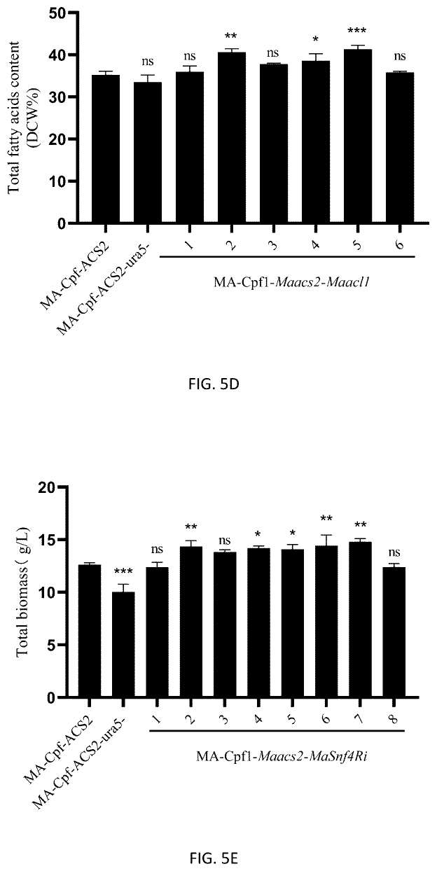

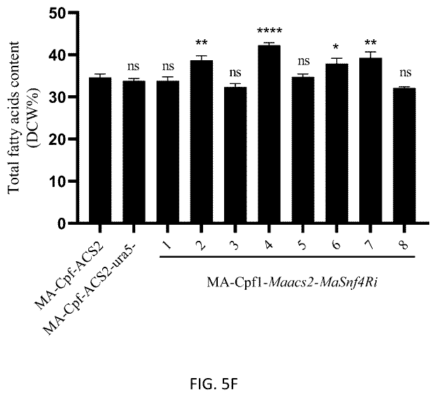

FIG. 5A-5F that the lipid accumulation level of the M. alpina was further improved by combining Maacs2 with other genes promoting the lipid accumulation of the M. alpina using a polygene operating system of CRISPR/Cpf1. Acetyl-CoA and reducing power NADPH are the most basic di-carbon unit and cofactor for fatty acid synthesis. By equilibrating the supply of the acetyl-CoA and the reducing power NADPH, synergistic expression of Maacs2 and g6pd2 improves the total biomass and lipid accumulation of the recombinant strain MA-Cpf1-Maacs2-Mag6pd2, and the total biomass of the 6 screened recombinant strains was improved by 7.18%-25.43% and can be up to 15.85 g/L (FIG. 5A, No. 6 transformant). The fatty acid content of the recombinant strains was also obviously improved, where the total fatty acid content of No. 2 transformant and No. 6 transformant was improved by 16.86% and 15.06% compared with that of the control group (FIG. 5B).

ACL is the most main source of cytoplasmic acetyl-CoA. An early experiment found that MaACL1 had a more remarkable effect on fatty acid accumulation of the M. alpina. Therefore, acl1 was further overexpressed on the basis of overexpressing Maacs2, so as to further improve the supply of the cytoplasmic acetyl-CoA. The fermentation result showed that the synergistic overexpression of the Maacs2 and acl1 obviously improved the biomass of the recombinant strain MA-Cpf1-Maacs2-Maacl1 up to 16.08 g/L (FIG. 5C, transformant No. 3), by 27.28% compared with the control group. The fatty acid content of the recombinant strains of transformants No. 2 and 5 was respectively improved by 15.21% and 17.22% (FIG. 5D) compared with the control group. The result indicated that the overexpression of the acl1 can promote the lipid synthesis of the strain by improving intracellular acetyl-CoA and further improve the lipid yield by obviously improving the biomass.

A sucrose non-fermenting kinase complex is a homologue of human AMP-activated protein kinase (AMPK). Both are heterotrimeric structures and inhibit the activity of ACC1, a key rate-limiting enzyme in inhibiting lipid synthesis through phosphorylation. It has been reported in the literature that knocking out or knocking down subunits of the SNF1 complex can promote lipid accumulation by increasing the ACC1 activity through de-phosphorylation on the ACC1. RNA interference (Masnf4Ri) was performed on a γ subunit of the SNF1 complex (coded by snf4) with unique gene coding of the M. alpina on the basis of MA-Cpf1-Maacs2. Meanwhile, the growth and lipid accumulation of the recombinant M. alpina MA-Cpf1-Maacs2-Masnf4Ri can be further promoted. The biomass content of each transformant was improved by 9.43%-17.10% compared with the control group (FIG. 5E). The total fatty acid content of No. 2 transformant was improved by 24.89% compared with the control group (FIG. 5F). The above case showed that by means of a CRISPR/Cpf1 polygene operating system, the multiplex combination and use of the Maacs2 gene were realized.

### Example 6 Combined Optimization of Synthesis and Utilization Pathways of M. alpina Acetyl-CoA

The specific steps were as follows:

The M. alpina MA-Cpf1-Maacs2-Maacl1 constructed in example 5 was used as a starting strain, induction and knockout of the screening marker ura5 gene were performed using a gene editing technique, and the step was the same as that described in example 5 to obtain knockout strain MA-Cpf1-Maacs2-Maacl1-Δura5.

The combined overexpression of Maacs2-Maacl1 can promote the supply of intracellular acetyl-CoA, but the failure to fully utilize acetyl-CoA over-supply can also lead to metabolic stress, such as high acetylation of proteins, so as to inhibit the lipid synthesis. In order to further optimize the acetyl-CoA utilization pathway, the genes described in example 5 were combined to perform RNA interference (Masnf4Ri) on a γ subunit of the SNF1 complex (coded by snf4) with unique gene coding of the M. alpina on the basis of MA-Cpf1-Maacs2-Maacl1, so as to protect the acetyl-CoA carboxylase activity and improve the availability of the acetyl-CoA. The experimental steps were the same as those in example 5 to obtain the recombinant strain MA-Cpf1-Maacs2-Maacl1-Masnf4Ri.

The result provided full theoretical support and technical methods for further improving the acetate tolerance of oleaginous microorganisms such as M. alpina by genetic engineering, improving the lipid accumulation capacity, and further developing oleaginous microorganism resources by establishment of an immunoblotting sample pretreatment method and use of a polygene system.

Although the present disclosure has been disclosed as above in the preferred examples, it is not intended to limit the present disclosure. Any person skilled in the art can make various changes and modifications without departing from the spirit and scope of the present disclosure. Therefore, the protection scope of the present disclosure should be as defined in the claims.

What is claimed is:

1. An acetic acid-tolerant recombinant strain, wherein the recombinant strain uses Mortierella alpina as a host, and recombinant strain overexpresses an acetyl-CoA synthetase with an amino acid sequence set forth in SEQ ID NO:1.

2. The recombinant strain according to claim 1, wherein the recombinant strain further knocks down or inhibits a γ subunit of a sucrose non-fermenting-related protein kinase, overexpresses a citrate lyase and/or overexpresses a glucose-6-phosphate dehydrogenase 2.

3. The recombinant strain according to claim 2, wherein the amino acid sequence of the citrate lyase is set forth in SEQ ID NO:9, an amino acid sequence of the glucose-6-phosphate dehydrogenase 2 is set forth in SEQ ID NO:10, and the amino acid sequence of the γ subunit of the sucrose non-fermenting-related protein kinase is set forth in SEQ ID NO:11.

4. The recombinant strain according to claim 1, wherein the recombinant strain takes a pBIG2-ura5s-ITs plasmid as an expression vector.

5. The recombinant strain according to claim 1, wherein the recombinant strain takes M. alpina MA-Pcbh1-LbCpf1-ura5⁻ or CCFM 501 as a host.

6. A method for regulating an oleaginous microorganism to synthesize a lipid, comprising using an ammonium salt as a nitrogen source, adding 0-30 mmol/L of an acetate, and overexpressing an acetyl-CoA synthetase with the amino acid sequence shown in SEQ ID NO:1 in the oleaginous microorganism.

7. The method according to claim 6, wherein the oleaginous microorganism comprises Mortierella alpina (M. alpina).

8. The method according to claim 6, wherein a concentration of the ammonium salt is 12-21 mmol/L and a concentration of the acetate is 12-30 mmol/L.

9. A method for synthesizing a lipid, comprising taking the recombinant strain according to claim 1 as a fermentation strain and synthesizing the lipid in a fermentation system containing an acetate and an ammonium salt.

10. The method according to claim 9, wherein the fermentation system contains 20-80 g/L of glucose, 1-2 g/L of a yeast extract, 5-10 g/L of potassium dihydrogen phosphate, 1.5-2.5 g/L of disodium hydrogen phosphate, 1-2 g/L of magnesium sulfate heptahydrate, 0.08-0.12 g/L of calcium chloride dihydrate and trace elements, 12-21 mmol/L of the ammonium salt, and 0-30 mmol/L of the acetate.

11. The method according to claim 9, wherein the ammonium salt comprises ammonium acetate and/or ammonium tartrate.

12. The method according to claim 9, wherein a concentration of the acetate is 12-30 mmol/L.

13. A method for extracting total protein of an oleaginous microorganism, comprising grinding the recombinant strain according to claim 1 under liquid nitrogen, adding precooled acetone containing 10% trichloroacetic acid to precipitate cells of the oleaginous microorganism, then adding a urea extracting solution, and finally adding iced acetone for precipitation to obtain total protein.


SEQUENCE LISTING (14 sequences)

SEQ ID NO: 1 - protein, 655 aa, Mortierella alpina
MSAEDTLFPVPARLLDPAQCPEPYVKSYEQYKELHRQSIENPDEFFGKMATELLSWSKPFHTVQHGGLEHGDIAWFLDGQLNAAYNCVDRHALADPNKIAIIYEADEPNESENITYSELLRQVCQLAGALRARGIRKGDTVAIYMPMIPEAIVAFLACARIGALHSVVFAGFSAEALRDRVQDAACRLVITADQGKRGGKTIETKKIVDDALKTCPSVETVIVCQRTGAEVPMKEGRDYWWNDECSKRMSYLTPEPMNSEDPLFLLYTSGSTGTPKGVMHTTAGYLLGAAATVKYVFDYHPGDIFACMADVGWITGHSYIVYGPLALGATTVLFESIPTYPTPARYWELIAKHKVTQFYTAPTAIRALRRLGDEWVKGHDLSSLRVLGTVGEPINPEAWHWYNDNVGKGRCAIVDTYWQTETGSHIVTPLPGAIPTKPGSATVPFFGIDIAILDPVTGKELVGNDVTGVLAVKKAWPSIARTVYNNHQRYLETYLKPYHGYYFTGDGATRDKDGYIWIRGRVDDVINVSGHRLSTAEIESALIMHHSVAETAVIGAPDDVTGQCIHAFVCLKPGVHCHKSDLSKELVLQVRKVIGPFAAPKKIYLVEDLPKTRSGKLMRRILRKIIAGEADSLGDISTLADPSVVEVLIAKVASS

SEQ ID NO: 2 - DNA, 1968 bp, Mortierella alpina
  source 1..1968: mol_type = genomic DNA
atgtctgctgaagatacccttttccccgtccccgccagactcctcgacccggcacagtgccccgagccctatgtcaagtcgtacgagcagtacaaggagctgcatcgccagagtatcgagaaccctgacgagttctttggcaagatggcaaccgagctgctttcctggtcaaagcctttccacacagtccagcacggtggacttgagcacggcgacattgcatggttcctggatggacagttgaacgcagcatacaactgtgtggatcgtcacgccttggctgatcccaacaagatcgccattatttacgaggctgatgaacccaacgagtccgagaacatcacatacagtgagctgcttcgtcaggtctgccagcttgctggtgccctgcgtgcccgcggcatccgcaagggcgacactgttgccatttacatgcccatgattcccgaggccatcgttgccttcctggcctgcgctcgcattggagctctccactcggtggtctttgccggtttctcggcagaggccctccgtgaccgtgtccaggacgccgcctgccgactggtcatcacggccgaccagggcaagcgcggtggaaagacgattgagaccaagaagattgtggacgacgctctcaagacgtgcccctcggtcgagaccgtgattgtttgccagcgcacgggtgctgaagtgccgatgaaggagggccgagactattggtggaacgacgagtgctccaagcgcatgagttacctcacccccgagcccatgaactctgaagaccccttgttcctcctctatacctccggctcgaccggcacgcccaaaggagtgatgcacaccactgcgggttaccttctcggcgctgcagccacggtcaagtacgtctttgactaccaccccggtgacatctttgcctgcatggccgatgtcggctggatcacaggacactcgtacattgtttatggacccttggctctgggtgccactaccgtcttgttcgagtccatcccaacataccccacccccgcccgctactgggagctgatcgccaagcacaaggtcacccagttctacaccgcgcccacagccatccgtgcccttcgtcgcctgggtgacgagtgggtgaagggtcatgacttgtcgagtctgcgcgtgctgggtactgtaggagagcccatcaaccccgaggcttggcactggtacaacgacaatgtcggcaagggccgctgcgctattgtggacacgtactggcagaccgagactggatcgcacattgtcacacctcttccaggagccattcccaccaagcctggttcagccacggtccccttctttggcattgacatcgccatcctcgaccctgtcactggaaaggagttggtcggcaacgatgtcactggagtgctggctgtcaagaaggcctggccctcgatcgcacgcacagtctacaacaaccaccagcgctatctggagacgtacctgaagccctaccacggatattactttacaggcgatggagcgacgcgcgacaaggacggatacatctggatccgtggccgtgtggacgatgtgattaatgtgtcgggacatcgtctctcgaccgccgagatcgagtcggcgctgatcatgcaccactccgtggccgagacggcagtcattggggcaccggacgatgtgacgggccagtgcatccacgcgtttgtgtgcctcaaaccaggtgtgcattgccacaagtccgatctcagcaaggaactcgtcctccaggtccgcaaggtcattggtccctttgcagcgcccaagaagatctacctggttgaggacctgcccaagactcggtctggaaaactgatgcgtcgcatcctgcgcaagatcattgcgggcgaggccgactccctgggcgatatctcgaccttggcggacccaagtgtcgtcgaggtcttgattgccaaagtcgccagctcttaa

SEQ ID NO: 3 - DNA, 63 bp, synthetic construct
  source 1..63: mol_type = other DNA
atggagcagaaactcatctctgaagaggatctggagcagaaactcatctctgaagaggatctg

SEQ ID NO: 4 - DNA, 51 bp, synthetic construct
  source 1..51: mol_type = other DNA
atggattacaaggatgacgacgataaggattacaaggatgacgacgataag

SEQ ID NO: 5 - DNA, 33 bp, synthetic construct
  source 1..33: mol_type = other DNA
cccaagcttatgtctgctgaagataccggctac

SEQ ID NO: 6 - DNA, 27 bp, synthetic construct
  source 1..27: mol_type = other DNA
tcccccgggttaagagctggcgacttt

SEQ ID NO: 7 - DNA, 23 bp, synthetic construct
  source 1..23: mol_type = other DNA
cacacacaaacctctctcccact

SEQ ID NO: 8 - DNA, 26 bp, synthetic construct
  source 1..26: mol_type = other DNA
caaatgaacgtatcttatcgagatcc

SEQ ID NO: 9 - protein, 1174 aa, synthetic construct
MSAKAVREYDGKLLLAHWLLRTPIPATSISATGSKFVQPATRLAHIGIDTAVLNTDKTVFNQHVQSLLDNLEQIHPWLLTAKLVAKPDQLIKRRGKSGLLLLNADWAEVRTWITAHAGKDVVVDSVAGVLKTFLVEPFIPHPANTEYYICINSDRDGDNILFTHEGGIEVGDVDAKALKLQVKVGDAFPTTTAIQTALLTHVPDSKHDVLVDFITRLYAVYVDLHFTYLEINPLVVLDPTPEHPAQVYYLDLAAKVDQTAEFEAGPKWAFARAHQNIGLVSAASQSVDAGPPMDFPAPFGRELTKEEAYVQELDSKTGASLKLTILNKDGRIWTMVAGGGASVVYSDAIAALGQANELANYGEYSGAPTETQTYEYAKTILDLMTRSAAPHPLGKVLIIGGGIANFTNVASTFKGIVRALTEFKQPLIAHKVRIFVRRGGPNYQEGLRSMRQLGETLGVEIQVFGPETHITEIVPLALTGKLSGLDQTGTATPSTHLSSGNFLQDQLLGNNTPHNAGSRASSPPPLEDRMTYFQDQNTEASESSHDENTPFTAHTRSFIYGMQPRAVQGMLDFDFICKREVPSVAAMVYPFGGAHVQKFYWGTKETLLPVFTSLDEAVAKYPEVDTVVNFASCRSVYDSTREIFKHSKHIRTISIIAEGVPERRARQILWEAKERNVLVIGPATVGGIKPGCFKIGNTGGMMDNIVSSKLYRAGSVAYVSKSGGMSNELNNIISRTTDGVYEGVAIGGDRYPGSTFIDHLLRYEKDPGCKMLVLLGEVGGVEEYKVCEAIKNGTIRKPVIAWCIGTCAKMFATEVQFGHAGALAQSDLETADAKNRALRAAGVIVPETFEMLPLVLSQTYQALVKKGVVTVRSEPETPKIPIDYSWAQELGLVRKPASFVSTICDDRGQELLYAGMRISDVFKEDIGIGGVLSLLWFKRRLPDYACKFIEMVLMLTADHGPAVSGAHNTIVTARAGKDLVSSLCAGLLTIGDRFGGALDGAAEQFSSAYDKSLSPREFVSSMRKQNKLILGIGHKIKSRTNPDLRVEIIKNYAKAHFPATPVLDYALAVETITTSKKDNLILNVDGAIGILFVDLLRNSGAFTREEAEEYIKIGTLNGLFVLGRTIGFIGHFLDQKRLKQGLYRHPWDDISYLTPGNELGRTVASLDSINKKAA

SEQ ID NO: 10 - protein, 514 aa, synthetic construct
MSEKKKHLYTVVVLGASGDLAKKKTFPALFGLFKNQYLDQNTHIVGYARTKMDRPKFEEHISKHIKLKTQDDKQALARFLKQCSYLDGQYDTDDGFQALEKSLVDIEKSSQATARLFYMALPPSVFIPVATNLKKHNYKKDALIRLIVEKPFGMDLESSRKLAKDLGALFTEEEIYRIDHYLGKEMVKNLMVLRFANVIFGSVWNRHCIDNVQITFKEKIGTEGRGGYFDEFGILRDVMLNHLLQILTLMTMEAPITLGAEDVRDEKVKVLRFIPPIAKEDVLLGQYTKSEDGKEPGYLDDKTVPKGSKTPTYAAATLYIHNERWSGVPFILKCGKALDQQKTEIRIQFKDVSGNLLSKLSRNEIVIRVQPKEAVYVKIMNKEPGLGMKTVISDLDLSYSSRFDSSVIPEAYESLILDSLNGDHSNFVRDDELDAAWKIFTPILKTIDNGEIVPVPYAFGSRGPKGVTEFVAKHGYMRGEQDYVWPQHSEAGANAKGQEQKVAKDQQKDTRTDH

SEQ ID NO: 11 - protein, 531 aa, synthetic construct
MSDNKDGTIASEPAETAAHGDAVQSGVDSHPQGSELMQGAPASEDASEEQPQGQSSTDYFSPQEPTVNAFTPQQQDDAVGFASEIGQPDQTTVNPEPDTTHPKDSGGLSNTQAPPASSLTAALNNNTNHSNTAGSDRPGPDAAPPGSATPSSQTTDVPATENIDPVASYSAWRKKKDTEMLQGTQAVMESFLKQHSAYDVLPVSYRQIVLDTTLLIKKALSVLMQYSIVSAPLWDSTKRKFAGMLTATDFINLIQFYYSHSSYTAAIHEMDQFQISQLRDVEKVIGVPPPELFSLHPLQSLYDACQLLVESRAHRLPLVDMDSETGEEMIVSVLTQYRILKFIAMNYQDEKTLRQPLSELKIGIYTDLAVADLSTPVMTVIDMFVKRRISSVPIVDEDGVVLNVFETVDVMTLVKAGVYRDLDLCVAEALLRRPEDFAGVHTCTLNDSLSSIFGTIRKSQVHRLIVVDSGNKLKGIVSLSDIMRYLIGGTRETKEPAGGNVEEAEVHVEDIEKETKSTHSGDGDDNDAGDL

SEQ ID NO: 12 - DNA, 3525 bp, synthetic construct
  source 1..3525: mol_type = other DNA
atgtctgccaaagctgttcgcgagtacgatggaaagctgctcttggcacactggctcctccgcacgcccatcccagcaaccagcatttctgccacaggatccaagtttgtccagcccgcaacgcgcctggcccacattggcattgataccgcagtgctgaacaccgacaagacggtcttcaaccagcacgtccagtccttgctcgataacctggagcagatccacccctggctcttgaccgccaagctcgtggccaagccagatcaattgatcaagcgccgtggcaagagcggcttgctgctgttgaacgcggactgggccgaggtcaggacctggattacagctcacgctggcaaggatgttgttgtcgactctgtcgccggagtgctcaagaccttcctggtcgagcccttcatccctcacccagccaacacggaatactacatctgcatcaactctgatcgcgatggcgataacattctcttcacacacgagggaggcattgaggtcggcgacgttgatgccaaggctttgaagcttcaggtcaaggtcggcgacgccttccccaccaccaccgcaattcagactgcgctgctcacacacgtccctgattccaagcacgatgtccttgtcgacttcatcacccgtctttacgccgtttacgttgatcttcacttcacctacctcgagatcaaccccttggtcgtcctcgaccccacccccgaacacccagcccaggtctactacttggatcttgcagccaaggtcgaccagactgcagagtttgaggctggccctaagtgggcctttgccagggcgcatcagaacattgggcttgtttctgctgcctctcaaagcgtggatgccggacctcctatggatttccctgctccttttggtcgtgagttgaccaaggaggaagcatatgttcaggagttggattccaagaccggtgcctcgctcaagctgacgattctgaacaaggacggtcgcatctggactatggtcgccggcggtggagcctcggttgtgtacagtgatgccattgctgccttaggacaggccaatgagctcgccaactacggagagtactctggagcacctaccgagacccagacttatgaatatgcaaagacgatactcgacttgatgactcgatctgccgctccccatcccctcggcaaggttctcatcattggaggtggtattgccaactttacgaacgtggcctcgaccttcaagggtattgtccgcgcgttgaccgagtttaagcagcctctgattgctcacaaggtgcgtattttcgtccgtcgcggaggccccaactatcaggagggtcttcgttctatgcgccagctgggtgagaccctgggagttgagattcaagtctttggtcccgagacccatatcaccgagattgtgcccttggccttgactggaaaactctctggacttgatcagactggtactgccacgcccagcacccatctgtcgtctggaaatttcctccaggaccagctcttgggcaacaacactccccataacgctggctcgcgagcatcttcaccaccaccattggaggaccggatgacttacttccaggaccagaacactgaggcttcagagtccagccacgacgagaacacaccctttacggcccacacgcgctcttttatttacggcatgcagccccgtgccgtccagggaatgcttgattttgatttcatttgcaagcgggaggtgccctcggtcgctgccatggtttaccccttcggaggggcccatgtccaaaagttttactggggcactaaagagacactcctgcctgtctttacttccttggacgaggctgtcgccaaataccctgaagtcgacactgtcgtgaactttgcctcgtgccgctccgtgtacgactccacccgcgagatctttaagcactccaagcatatccgcaccatctccatcattgccgagggtgtgccagagcgccgcgctcgtcagatcttgtgggaggccaaggaacgtaatgttttggttattggacctgccaccgttggaggcatcaagcctggctgcttcaagattggcaacactggaggaatgatggacaacattgtgtcttcgaagctttaccgcgcaggatccgtggcgtacgtgtccaaatccggaggcatgtctaacgagttgaacaacattatctctcgtaccaccgacggtgtctacgagggagttgccatcggaggggaccgttaccctggttcgacttttatcgaccacctgcttcgctacgagaaggaccctggctgcaagatgcttgtcctgttgggcgaagtcggcggtgttgaagagtacaaagtctgtgaagcgatcaagaacggaaccatccgcaagcccgtgattgcttggtgcattggtacctgcgccaagatgtttgccaccgaggtccagtttggacacgctggggcccttgcacagtcagaccttgagactgctgatgccaagaaccgcgcccttcgtgctgctggagtgatcgtcccagagacgtttgagatgcttcctctggttttgagccagacctatcaggctctcgtcaagaagggcgtcgtcactgtccgctctgagcccgagacacccaagatccccattgattactcttgggcccaggagttggggcttgtccgcaagcctgcctcgtttgtgtcgaccatctgtgatgaccgtggccaggaactgctctatgctggcatgcgtatctcggatgtgttcaaggaggacattggcatcggaggagtcctctcgctgctctggttcaagcgccgtctccctgactatgcctgcaagtttatcgagatggtcctcatgcttactgctgatcacggtcccgccgtctcgggtgcgcataacacgattgtgaccgctcgcgctggcaaggatcttgtctcgtcattgtgcgcgggtctgttgacgatcggagaccgttttggaggtgcattggacggagccgctgagcagttctcgtctgcctacgacaagtctctctcgccccgcgagtttgtgtcgtcgatgagaaagcaaaacaagctgatcttgggtatcggtcacaagatcaagtcgcgcacgaaccctgatttgcgcgtggagatcatcaagaactacgccaaggcacatttccctgccacgcctgtgctggactatgccctggctgtggagacgatcaccacctccaagaaggataacttgatcctgaacgtggacggagccatcggtatcctgtttgtggacttgctgagaaactcgggagcgttcacgcgcgaggaagcagaggagtacatcaagattggaaccttgaatggtctctttgtcctgggacggacgattggattcattggacactttttggatcagaagaggctgaagcaagggctgtacagacacccttgggatgatatctcgtacctgacgcctggcaacgagctcggacggacagttgcctcgttggactctatcaacaagaaggctgcctaa

SEQ ID NO: 13 - DNA, 1545 bp, synthetic construct
  source 1..1545: mol_type = other DNA
atgtctgagaagaagaagcatctttacactgtggttgtgctcggtgcatcgggagacctagccaagaagaaaaccttcccggcgctctttggactcttcaaaaaccaatatctggatcaaaacacgcacatcgtcggctatgcgcgtacaaagatggatcggcccaagtttgaggagcacatctcaaaacacatcaaactcaagacccaagacgacaagcaggcactcgcccgctttctcaaacaatgttcttacctcgacggacagtatgacacagacgacggctttcaagcgctcgaaaagtcacttgtggatattgaaaagtcctcgcaggccaccgcacggctgttctacatggccttgcctccctcagtgtttattccagtagccacgaacctcaagaagcacaactacaaaaaggacgcgctcattcggctgatcgtcgaaaagccctttggaatggatctcgagagctcgcgcaaattggccaaagatctaggcgccctcttcaccgaggaagagatttatcgcatcgaccactacctgggcaaggagatggtcaaaaatttgatggtcctccgcttcgccaatgtcatcttcggttccgtatggaatcggcattgcatcgataatgttcaaatcacttttaaggaaaagattggcactgagggcagaggcggttactttgacgagtttggtatccttcgcgacgttatgctaaaccacttgctgcagattcttacgctgatgacgatggaagcccccatcacgttgggcgccgaggatgtgcgggacgaaaaggtcaaagtcctccgcttcatcccacccatcgcgaaggaggatgttttattaggccagtacaccaagagtgaagacggcaaggagcccggctatctcgatgacaagacagttcccaagggtagtaagaccccgacttatgcggccgccacgctctacatccataacgagcgctggtccggtgtgcccttcatcctcaaatgtggcaaagccctcgaccagcaaaagaccgagatccgtatccagttcaaggacgtctctggaaacctgctctcgaagctgtccaggaacgagatcgttatccgggtccagcccaaggaggccgtctatgtcaagatcatgaacaaggagccaggcttgggtatgaagaccgtcatctctgaccttgacctctcgtactcgagccgattcgacagctctgtcatccctgaggcctacgagtctttgatcttggactctttgaacggagatcactccaacttcgtccgtgacgatgaactggacgcggcctggaagatcttcacgcccattctcaagacgatcgacaatggtgaaattgtgccggttccttatgcgtttggaagccgaggtcccaagggcgtgacagagttcgtggccaagcacggctatatgcgtggagagcaagattacgtctggccgcagcattctgaggccggtgccaacgccaaggggcaagagcagaaggtggcgaaggaccagcaaaaggacacaaggactgaccattaa

SEQ ID NO: 14 - DNA, 1596 bp, synthetic construct
  source 1..1596: mol_type = other DNA
atgagcgacaacaaggacggtaccattgcttccgagccagctgaaacagccgcgcatggggacgcggtgcaaagcggtgttgactctcacccacaagggtcagagctaatgcaaggagcaccggcatcagaggatgccagcgaggagcaaccgcagggccagtcctcaaccgactacttttcgcctcaagaacccacagtcaatgcttttactccgcaacagcaggacgacgctgtggggtttgcgtcagaaataggccagcctgatcaaacaacagtgaaccccgaacccgataccactcaccctaaagactcaggaggcctgtcgaatacgcaagcacccccggcctcgtcgctgacggcagccctcaacaataacaccaaccactctaatacggctgggtctgacaggccgggaccggatgctgcaccaccaggatcggctaccccatcctcacaaacaacggatgtgcctgcgacggaaaatattgaccctgttgcatcgtactctgcatggcgcaagaagaaagacacggaaatgttgcaaggaacccaggctgtgatggagtcgtttctaaagcagcactccgcctacgatgtgttacccgtcagctatcggcaaattgttttggataccacgctcttgatcaagaaggccctttcggtcctgatgcagtacagcatagtgtctgctccgttgtgggattcaaccaagcgcaagtttgcaggcatgctgacggccacagactttatcaatttgattcagttttactactcccattcatcctacaccgccgcaattcatgagatggaccaattccagatatcccagctcagagatgtcgagaaggtaataggagtgccaccgcccgagctgttctcgctacacccactgcaatcgctctacgacgcctgtcagttgctggtcgaatcaagagcgcatcggttacccttggtggatatggactctgaaacaggagaggagatgatcgtgtcagtcttgactcagtacagaatcctcaagttcattgccatgaactatcaggacgagaagacacttcgtcaaccgctttcggagctcaagattgggatctatacggaccttgccgtggcagatctttccacgcccgtgatgaccgtgattgacatgttcgtgaagcggaggatatcctctgtgcccattgtcgatgaggacggtgtggttctgaacgtctttgagactgtcgatgtgatgacgctggtcaaggcaggcgtgtacagggatttggatctctgcgtcgcagaggcgttactgcggagaccggaagactttgcaggagttcatacctgcacgctcaacgacagcctctcgtccattttcggaaccattcgcaagtctcaagtccatcgcctgatcgtcgttgacagcggcaataagctgaagggaatcgtcagtctctcggatatcatgcgttatctcatcggtggaacacgtgagaccaaggagcctgcaggtggcaatgtcgaagaggcggaagtccacgtcgaggacatcgagaaggaaaccaaatcgacgcacagtggcgatggcgatgataatgatgcgggagacctatga